(19) United States
(12) Patent Application Publication
(10) Pub. No.: US 2017/0273737 A1
(43) Pub. Date: Sep. 28, 2017

(54) CONNECTION STRUCTURE AND CONNECTION METHOD

(71) Applicant: OLYMPUS CORPORATION, Tokyo (JP)

(72) Inventors: Takayoshi IWANAMI, Tokyo (JP); Yuya HIDAKA, Tokyo (JP)

(73) Assignee: OLYMPUS CORPORATION, Tokyo (JP)

(21) Appl. No.: 15/620,313

(22) Filed: Jun. 12, 2017

Related U.S. Application Data
(63) Continuation of application No. PCT/JP2015/076879, filed on Sep. 24, 2015.

(30) Foreign Application Priority Data
Dec. 26, 2014 (JP) 2014-264867

(51) Int. Cl. A61B 18/14; H01R 43/048; H01R 4/18

(52) U.S. Cl. CPC A61B 18/1482; H01R 4/183; H01R 43/048; A61B 2018/1405; A61B 2018/00601

(57) ABSTRACT

Provided is a connection structure for connecting a treatment component to an operating wire using a coupling member. The treatment component has a rod-shaped proximal end. The coupling member includes a first hole portion into which the proximal end is configured to be inserted on one end, and a second hole portion into which the operating wire is configured to be inserted on the other end. With the proximal end inserted into the first hole portion, the proximal end and the coupling member are joined by swaging and plastically deforming part of the first hole portion. With the operating wire inserted into the second hole portion, the operating wire and the coupling member are joined by swaging and plastically deforming part of the second hole portion. Joint strength between the proximal end and the coupling member is higher than joint strength between the operating wire and the coupling member.

### CROSS REFERENCES TO RELATED APPLICATIONS

This application is a continuation of PCT international application Ser. No. PCT/JP2015/076879, filed on Sep. 24, 2015 which designates the United States, incorporated herein by reference, and which claims the benefit of priority from Japanese Patent Application No. 2014-264867, filed on Dec. 26, 2014, incorporated herein by reference.

### BACKGROUND

### 1. Technical Field

The disclosure relates to a connection structure and a method for connecting a treatment component to an operating wire in an endoscopic treatment tool.

### 2. Related Art

Conventionally, high frequency knifes have been known as an example of a treatment tool (hereinafter, also referred to as endoscopic treatment tool) for removal of tissue such as a mucosa in endoscopic surgery (refer to JP-A 2004-248911, JP-A 63-97154, and JP-UM-A 62-50610). For example, JP-A 2004-248911 discloses a structure of a high frequency knife including an insertion portion to be inserted into a living body and an operating unit connected to the proximal end portion of the insertion portion (refer to FIG. 1 of JP-A 2004-248911). The insertion portion is formed by a flexible sheath, a stopper member provided at the distal end portion of the flexible sheath, and an insulating tip. The outer peripheral portions of these members are covered with an insulating tube. An operating wire is inserted into the insertion portion so as to be movable in the axial direction. The proximal end side of the operating wire is connected to a wire operating handle for operating the operating wire. On the other hand, an electrode portion serving as an incision tool (knife) as a treatment component extends from the distal end portion of the operating wire in the axial direction and is fixed thereto via a coupling member. The coupling member is provided with a stopper receiving portion, and as the stopper receiving portion abuts on the stopper member, the protruding length of the electrode portion is restricted. In addition, JP-A 2004-248911 discloses a structure of an electrode portion of which the distal end has a plate shape with a plurality of corner portions (hook portions) (refer to FIG. 2 and the like in JP-A 2004-248911).

In such an endoscopic treatment tool, in order to connect members (for example, an operating wire and an electrode portion) to each other, hitherto, brazing which achieves high joint strength and enables electrical connection has been used. For example, JP-A 63-97154 discloses a technique for connecting a rod-shaped electrode to an operating wire by inserting the rod-shaped electrode and the operating wire respectively from both ends of a tubular coupling member provided with a brazing filler material injection hole at the side surface thereof, and injecting the brazing filler material into the brazing filler material injection hole (refer to FIG. 5 of JP-A 63-97154).

### SUMMARY

In some embodiments, provided is a connection structure for connecting a treatment component to an operating wire using a coupling member in an endoscopic treatment tool. The treatment component has a rod-shaped proximal end portion. The coupling member includes a first hole portion into which the rod-shaped proximal end portion is configured to be inserted on one end side of the coupling member, and a second hole portion into which the operating wire is configured to be inserted on the other end side of the coupling member. With the rod-shaped proximal end portion inserted into the first hole portion, the rod-shaped proximal end portion and the coupling member are joined together by swaging and plastically deforming at least a part of a peripheral wall of the first hole portion. With the operating wire inserted into the second hole portion, the operating wire and the coupling member are joined together by swaging and plastically deforming at least a part of a peripheral wall of the second hole portion. A joint strength between the rod-shaped proximal end portion and the coupling member is higher than a joint strength between the operating wire and the coupling member.

In some embodiments, provided is a method for connecting a treatment component having a rod-shaped proximal end portion to an operating wire in an endoscopic treatment tool using a coupling member, the coupling member having, on one end side thereof, a first hole portion into which the rod-shaped proximal end portion is configured to be inserted and having, on the other end side thereof, a second hole portion into which the operating wire is configured to be inserted. The method includes: inserting the rod-shaped proximal end portion into the first hole portion, and swaging and plastically deforming at least a part of a peripheral wall of the first hole portion to join the rod-shaped proximal end portion and the coupling member together; and inserting the operating wire into the second hole portion, and swaging and plastically deforming at least a part of a peripheral wall of the second hole portion to join the operating wire and the coupling member together in such a way that a joint strength between the operating wire and the coupling member is smaller than a joint strength between the rod-shaped proximal end portion and the coupling member. Joining of the rod-shaped proximal end portion and the coupling member together and joining of the operating wire and the coupling member together are performed in an arbitrary order.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic external view of an endoscopic treatment tool (high frequency knife) to which a connection structure according to a first embodiment of the present invention is applied;

FIG. 2 is an enlarged partial sectional view illustrating the distal end portion of an insertion portion illustrated in FIG. 1;

FIG. 3 is a plan view of the insertion portion illustrated in FIG. 2, when viewed from the distal end side;

FIG. 4 is a schematic view illustrating another example of the structure of an electrode portion;

FIG. 5 is a schematic view illustrating still another example of the structure of the electrode portion;

FIG. 6 is a schematic view illustrating still another example of the structure of the electrode portion;

FIG. 7A is a schematic view for describing the connection structure and a connection method according to the first embodiment of the present invention;

FIG. 7B is a schematic view for describing the connection structure and the connection method according to the first embodiment of the present invention;

FIG. 8A is a schematic view for describing the connection structure and the connection method according to the first embodiment of the present invention;

FIG. 8B is a schematic view for describing the connection structure and the connection method according to the first embodiment of the present invention;

FIG. 9 is a perspective view illustrating a state in which a rod-shaped electrode portion and an operating wire are connected via a coupling member;

FIG. 10 is a schematic view for describing a method of setting swaging conditions using a swaging diameter;

FIG. 11 is a graph illustrating a relationship between a swaging amount and a joint strength in the connection structure according to the first embodiment of the present invention;

FIG. 12 is a sectional view illustrating swaging dies that can be used in the first embodiment of the present invention;

FIG. 13 is a sectional view illustrating swaging dies that can be used in the first embodiment of the present invention;

FIG. 14A is a schematic view for describing a connection structure and a connection method according to a second embodiment of the present invention;

FIG. 14B is a schematic view for describing the connection structure and the connection method according to the second embodiment of the present invention; and

FIG. 15 is a graph illustrating a relationship between a swaging amount and a joint strength in the connection structure according to the second embodiment of the present invention.

### DETAILED DESCRIPTION

Hereinafter, embodiments of a connection structure and a connection method according to the present invention will be described in detail with reference to the drawings. The present invention is not limited by these embodiments. The reference signs are used to designate the same elements throughout the drawings. The drawings are schematic and relationships and ratios between the dimensions of the components are different from reality. The relationships and ratios between the dimensions of the elements may vary between drawings.

### First Embodiment

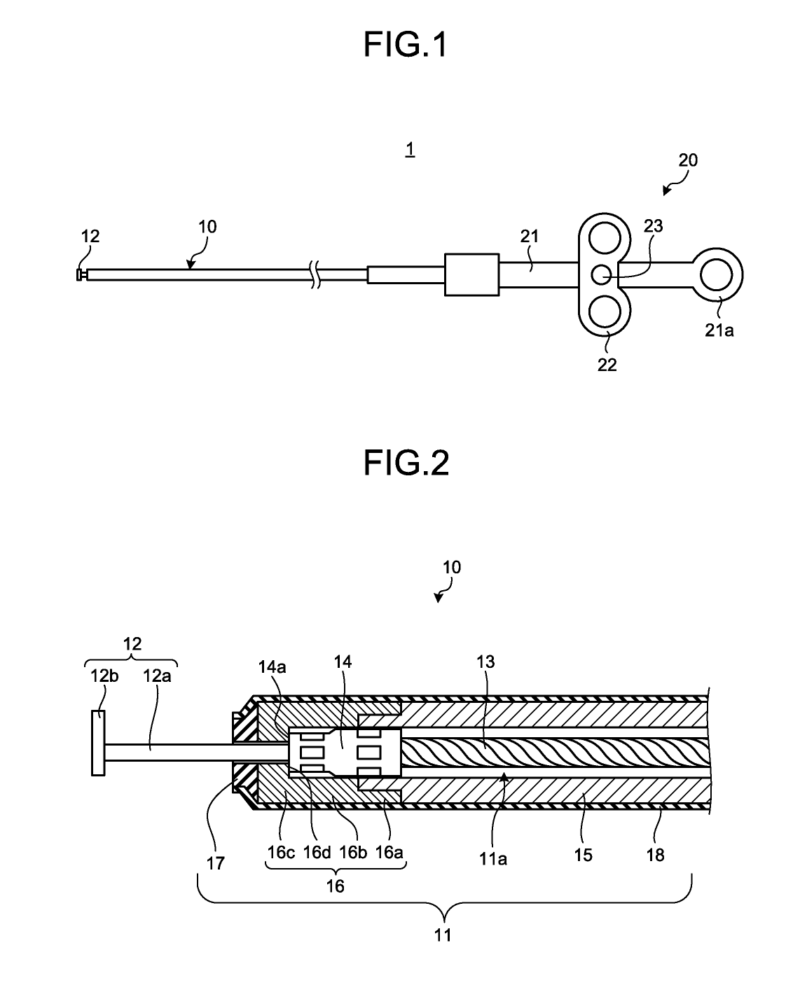

FIG. 1 is a schematic external view of a high frequency knife as an example of an endoscopic treatment tool to which a connection structure according to a first embodiment of the present invention is applied. A high frequency knife 1 illustrated in FIG. 1 includes an insertion portion 10 configured to be inserted into a treatment tool channel of an endoscope, and an operating unit 20 provided at the proximal end of the insertion portion 10. At the distal end of the insertion portion 10, an electrode portion (a knife portion) 12 is provided, as an example of a treatment component, to remove tissue by high frequency current.

The operating unit 20 includes an operating unit body 21 in which a support portion 21a is provided at an end portion of a long and thin tube, and a wire operating handle 22 which is slidable in the axial direction with respect to the operating unit body 21. The wire operating handle 22 is provided with a connector portion 23 to which a cord extending from a high frequency generator for supplying a high frequency current to the electrode portion 12 is electrically connected.

FIG. 2 is an enlarged partial sectional view illustrating the distal end portion of the insertion portion 10 illustrated in FIG. 1. As illustrated in FIG. 2, the insertion portion 10 includes a flexible sheath 11, the electrode portion 12 provided so as to protrude from and retract into the distal end of the flexible sheath 11, an operating wire 13 which is inserted into the flexible sheath 11 and is connected to the electrode portion 12, and a coupling member 14 which connects the electrode portion 12 to the operating wire 13. In FIG. 2, only the flexible sheath 11 is shown in a section.

The flexible sheath 11 includes, for example, a close-wound coil 15 formed in a tubular shape by winding a metal in a close coil shape, a stopper member 16 provided at the distal end of the close-wound coil 15, an insulating tip 17 provided at the distal end of the stopper member 16, and an insulating tube 18 for covering the outer peripheries of the close-wound coil 15, the stopper member 16, and the insulating tip 17.

The stopper member 16 is provided with a fitting portion 16a which has a tubular shape with a uniform outer diameter and is fitted to the distal end of the close-wound coil 15, an insertion portion 16b having an inner diameter into which the coupling member 14 is configured to be inserted, and a thick portion 16c into which a rod-shaped electrode portion 12a on the proximal end side of the electrode portion 12 is configured to be inserted and which has an inner diameter smaller than that of the insertion portion 16b.

The insulating tip 17 is a ring-shaped insulating member provided with an opening through which the rod-shaped electrode portion 12a is configured to be inserted. The opening diameter of the insulating tip 17 is substantially the same as the inner diameter of the thick portion 16c of the stopper member 16 and is provided so that the inner peripheral surfaces thereof are continuous.

The insulating tube 18 is formed of a resin material such as a tetrafluoroethylene material and integrally covers the close-wound coil 15, the stopper member 16, and the insulating tip 17.

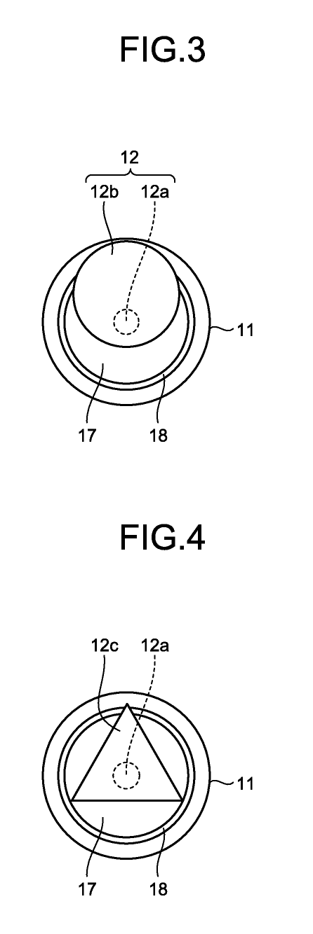

The electrode portion 12 includes the rod-shaped electrode portion 12a having a rod shape, and a plate-shaped electrode portion 12b provided at the distal end of the rod-shaped electrode portion 12a. The plate-shaped electrode portion 12b is provided in a direction intersecting the longitudinal direction of the rod-shaped electrode portion 12a. FIG. 3 is a plan view of the insertion portion 10 viewed from the distal end side. As illustrated in FIGS. 2 and 3, the plate-shaped electrode portion 12b has a disk shape with a diameter larger than that of the rod-shaped electrode portion 12a. The rod-shaped electrode portion 12a and the plate-shaped electrode portion 12b are integrally formed of a conductive material such as stainless steel (for example, SUS304) by, for example, cutting work.

Alternatively, the rod-shaped electrode portion 12a and the plate-shaped electrode portion 12b may be formed of different materials. For example, the rod-shaped electrode portion 12a may be formed of a conductive material such as stainless steel and, instead of the plate-shaped electrode portion 12b, a plate-shaped member formed of an insulating material such as ceramics may join to the rod-shaped electrode portion 12a.

In addition, the planar shape of the plate-shaped electrode portion 12b is not limited to a circular shape, and a plate-shaped electrode portion 12c having a triangular shape as illustrated in FIG. 4 may be provided. Otherwise, a plate-shaped electrode portion having a plurality of corner portions like a polygonal shape with four or more corners or a star shape may also be provided.

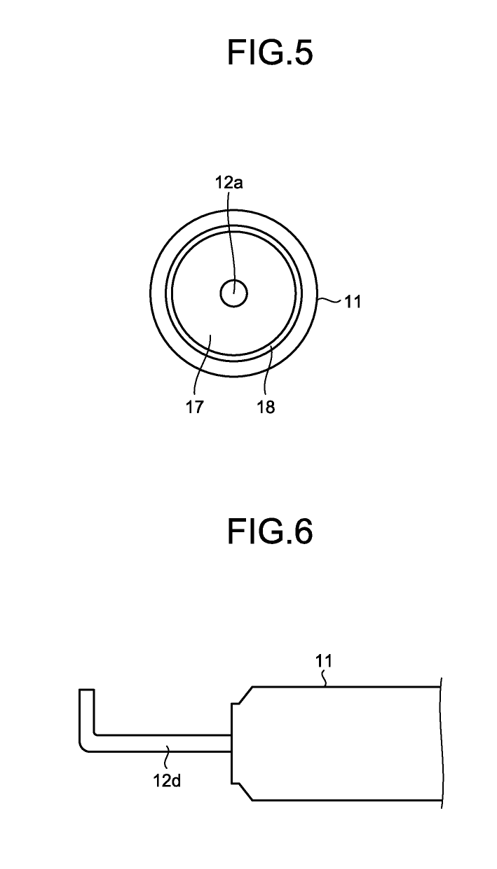

Alternatively, as illustrated in FIG. 5, the electrode portion may be composed only of the rod-shaped electrode portion 12a, without providing the plate-shaped electrode portion 12b.

Furthermore, as illustrated in FIG. 6, instead of the rod-shaped electrode portion 12a and the plate-shaped electrode portion 12b illustrated in FIG. 3, a hook-shaped electrode portion 12d in which the distal end of a rod-shaped electrode member is bent in an L shape may be provided.

The operating wire 13 is a strand wire (with, for example, 7 strands) made of a conductive material such as stainless steel (for example, SUS304) and is inserted through an insertion hole 11a provided in the flexible sheath 11 so as to be movable in the axial direction. The operating wire 13 is electrically connected to the electrode portion 12 on the distal end side and is electrically connected to the connector portion 23 illustrated in FIG. 1 on the proximal end side.

The electrode portion 12 and the operating wire 13 are electrically connected to each other via the coupling member 14, and each joins to the coupling member 14. The coupling member 14 is a tubular member made of a conductive material such as stainless steel (for example, SUS304) and is produced from a rod material or a tube material. The rod-shaped electrode portion 12a and the operating wire 13 are inserted into the coupling member 14 so as to cause the end faces thereof to face each other. Each of the regions in which the rod-shaped electrode portion 12a and the operating wire 13 are inserted is swaged from an outer periphery of the coupling member 14, so that the electrode portion 12, the operating wire 13, and the coupling member 14 are integrated together. The connection structure between the electrode portion 12 and the operating wire 13 using the coupling member 14 will be described later in detail.

With the coupling member 14 provided inside the flexible sheath 11, the coupling member 14 is movable along the axial direction between the position where the plate-shaped electrode portion 12b abuts on the distal end face of the insulating tip 17 and the position where a distal end face 14a of the coupling member 14 abuts on an inner bottom surface 16d of the insertion portion 16b of the stopper member 16. The amount of protrusion of the electrode portion 12 from the insertion portion 10 is restricted by the distal end face 14a of the coupling member 14 abutting on the inner bottom surface 16d of the stopper member 16.

Returning to FIG. 1, an insertion hole (not illustrated) through which the operating wire 13 is inserted is provided in the operating unit body 21. The operating wire 13 inserted into the flexible sheath 11 further extends to the proximal end side of the operating unit body 21 through the insertion hole in the operating unit body 21 and is connected to the wire operating handle 22. By causing the wire operating handle 22 to slide in the axial direction, the operating wire 13 advances and retreats in the axial direction inside the insertion hole 11a of the flexible sheath 11 such that the electrode portion 12 protrudes from and retracts into the distal end portion of the insertion portion 10. In addition, by connecting the high frequency generator to the connector portion 23 and generating a high frequency current, the high frequency current is supplied to the electrode portion 12 through the connector portion 23, the operating wire 13, and the coupling member 14. This allows the electrode portion 12 to remove tissue.

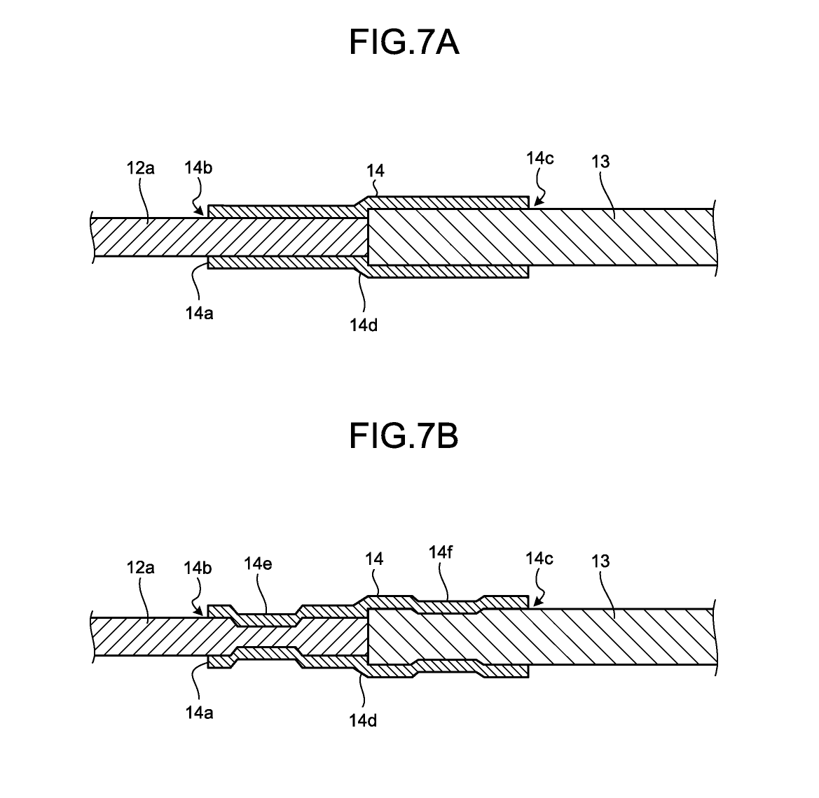

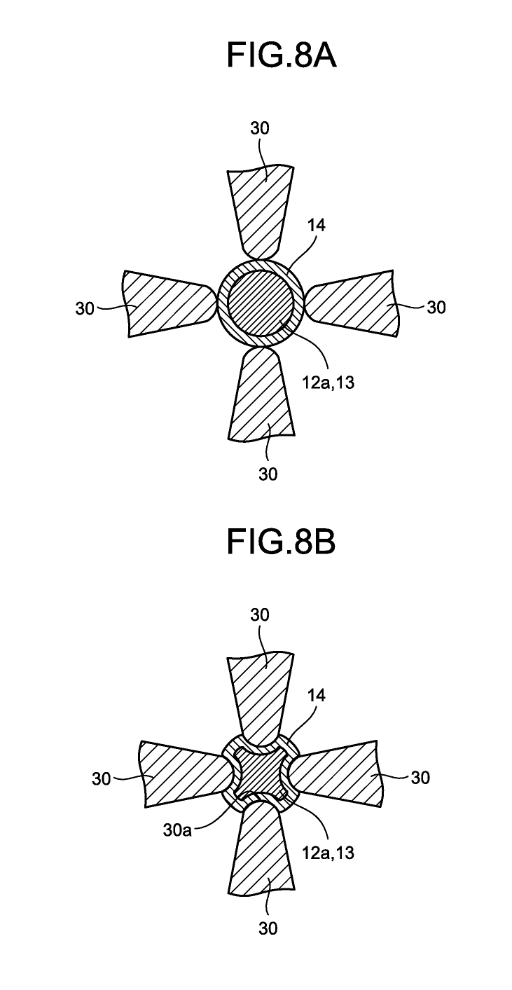

Next, the connection structure between the electrode portion 12 and the operating wire 13 using the coupling member 14 and a connection method will be described. FIGS. 7A, 7B, 8A, and 8B are schematic views for describing the connection structure and the connection method according to the first embodiment. FIG. 7A illustrates a state in which the rod-shaped electrode portion 12a and the operating wire 13 are inserted into the coupling member 14 (before connection). FIG. 7B illustrates a state in which the rod-shaped electrode portion 12a, the operating wire 13, and the coupling member 14 are integrated together by swaging the coupling member 14 (after connection).

Here, the dimensions of the electrode portion 12 and the operating wire 13 vary depending on the application and the like of the high frequency knife 1. In the following description, as an example, a case where the rod-shaped electrode portion 12a has an outer diameter of about 0.4 mm and a length of about 10 mm and the operating wire 13 has an outer diameter of about 0.5 mm will be described.

As illustrated in FIG. 7A, the coupling member 14 is a substantially cylindrical member in which an electrode insertion portion 14b into which the rod-shaped electrode portion 12a is configured to be inserted and a wire insertion portion 14c into which the operating wire 13 is configured to be inserted are provided at both ends. The electrode insertion portion 14b and the wire insertion portion 14c may or may not communicate with each other inside the coupling member 14. Preferably, the two may communicate with each other, and the end face of the rod-shaped electrode portion 12a and the end face of the operating wire 13 may abut on each other. Accordingly, the total length of the coupling member 14 decreases and can smoothly advance or retreat even in a state where the flexible sheath 11 is curved.

In the case where the dimensions of the electrode portion 12 and the operating wire 13 are as mentioned above, it is preferred that, for example, the coupling member 14 has a length of about 5 mm, the electrode insertion portion 14b has an inner diameter of about 0.43 mm, a peripheral wall thickness of about 0.185 mm, an outer diameter of about 0.8 mm, and a length of about 2.5 mm, and that the wire insertion portion 14c has an inner diameter of about 0.53 mm, a peripheral wall thickness of about 0.185 mm, an outer diameter of about 0.9 mm, and a length of about 2.5 mm.

As illustrated in FIG. 7A, in a case where the outer diameter of the rod-shaped electrode portion 12a is smaller than the outer diameter of the operating wire 13, when the thickness of the peripheral walls of the electrode insertion portion 14b and the wire insertion portion 14c are caused to be substantially uniform, a stepped portion is formed on the outer periphery of the coupling member 14. In this case, in order to prevent the stepped portion from being caught on the inner peripheral surface of the close-wound coil 15 or the stopper member 16 when the operating wire 13 is advanced or retreated, it is preferable that a tapered portion 14d is provided in the stepped portion to smoothen the outer peripheral shape. The outer diameter of the rod-shaped electrode portion 12a and the outer diameter of the operating wire 13 may have almost the same dimensions. In this case, when the thicknesses of the peripheral walls of the electrode insertion portion 14b and the wire insertion portion 14c are caused to be substantially uniform, the coupling member 14 has a cylindrical shape having uniform outer diameter dimensions without the stepped portion. That is, the shape of the coupling member 14 is not particularly limited.

To connect the rod-shaped electrode portion 12a to the operating wire 13, first, the operating wire 13 is inserted into the wire insertion portion 14c of the coupling member 14. Subsequently, as illustrated in FIG. 8A, swaging dies 30 abut on the outer periphery of the coupling member 14 which is the peripheral wall of the wire insertion portion 14c, and are pressed against the center axis of the coupling member 14 as illustrated in FIG. 8B (swaging process).

In the swaging process, a general-purpose swaging tool used for swage connection between a connector pin for electrical wiring and an electric wire may be used. The four swaging dies 30 illustrated in FIGS. 8A and 8B are schematic views illustrating a part of a 4-indent swaging tool as an example of the general-purpose swaging tool. The swaging dies 30 are installed to be able to advance and retreat on an axis orthogonal to the center axis of members to be joined (in FIGS. 8A and 8B, the coupling member 14 and the operating wire 13, or the coupling member 14 and the rod-shaped electrode portion 12a). Furthermore, the swaging dies 30 are arranged at equal intervals so as to cause distal end portions 30a thereof to be located on a circumference having the same distance from the center axis of the members to be joined, and are configured to advance and retreat at the same time.

By pushing the swaging dies 30 abutting on the outer periphery of the coupling member 14 toward the center axis by a predetermined amount, the peripheral wall of the coupling member 14 is plastically deformed in the radially inward direction to press the operating wire 13. Thereby, the operating wire 13 is also plastically deformed by the pressing force. As a result, the coupling member 14 and the operating wire 13 are brought into close contact with each other and are joined together.

Subsequently, the rod-shaped electrode portion 12a is inserted into the electrode insertion portion 14b of the coupling member 14, and as illustrated in FIG. 8A, the swaging dies 30 abut on the outer periphery of the coupling member 14 which is the peripheral wall of the electrode insertion portion 14b. Then, as illustrated in FIG. 8B, the swaging dies 30 are pressed against the center axis of the coupling member 14 so as to be pushed by a predetermined amount (swaging process). Accordingly, the peripheral wall of the coupling member 14 is plastically deformed in the radially inward direction to press the rod-shaped electrode portion 12a. Thereby, the rod-shaped electrode portion 12a is also plastically deformed by the pressing force. As a result, the coupling member 14 and the electrode portion 12 are brought into close contact with each other and are joined together. Either the electrode portion 12 or the operating wire 13 may join to the coupling member 14 first.

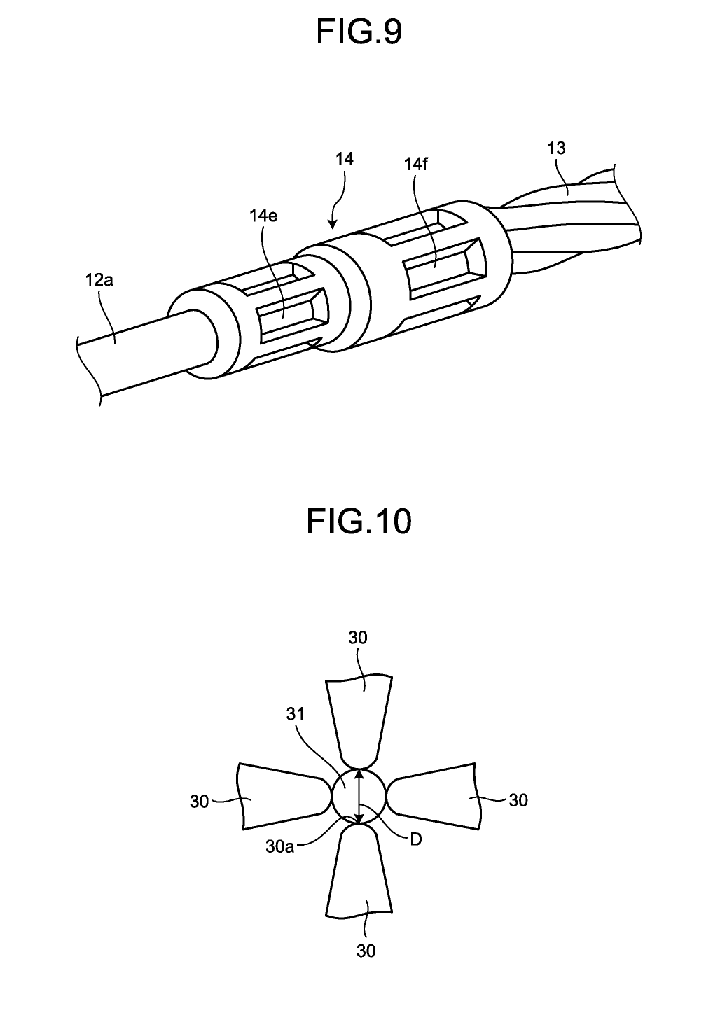

FIG. 9 is a perspective view illustrating a state in which the rod-shaped electrode portion 12a and the operating wire 13 are connected via the coupling member 14. On the outer periphery of the coupling member 14, indents 14e and 14f are formed which are recessed due to the plastic deformation of the parts where the swaging dies 30 have been abutted.

In the above-described swaging process, processing conditions (swaging conditions) are specified so that the joint strength between the rod-shaped electrode portion 12a and the coupling member 14 and the joint strength between the operating wire 13 and the coupling member 14 respectively are equal to predetermined joint strengths. The swaging conditions can be set by the diameter (swaging diameter) of the circumference through which the distal end portions 30a pass when the four swaging dies 30 are pushed during the swaging, or the pushing amounts of the distal end portions 30a in the radial direction from the outer periphery of the member to be joined during the swaging.

FIG. 10 is a schematic view for describing a method of setting the swaging conditions using the swaging diameter. In this case, using a pin gauge 31 having a diameter D, the position of each of the swaging dies 30 is accurately adjusted so that the distal end portions 30a of the four swaging dies 30 come into contact with the circumference of the pin gauge 31, and the positions are determined as the final positions of the swaging dies 30. When the swaging is performed, the swaging dies 30 may be moved in the radial direction until the distal end portions 30a of the swaging dies 30 reach the final positions.

In practice, however, even if the swaging diameters are the same, the amount of deformation of the member to be joined may vary during the swaging depending on the outer diameter dimensions of the member to be joined. In such a case, the swaging conditions may be set by the swaging amount. Specifically, the difference obtained by subtracting the swaging diameter from the outer diameter of the member to be joined before processing is used as the swaging amount. In this case, when the swaging is performed, the swaging dies 30 may be moved in the radial direction by the swaging amounts from the positions where the distal end portions 30a of the swaging dies 30 are in contact with the outer periphery of the member to be joined.

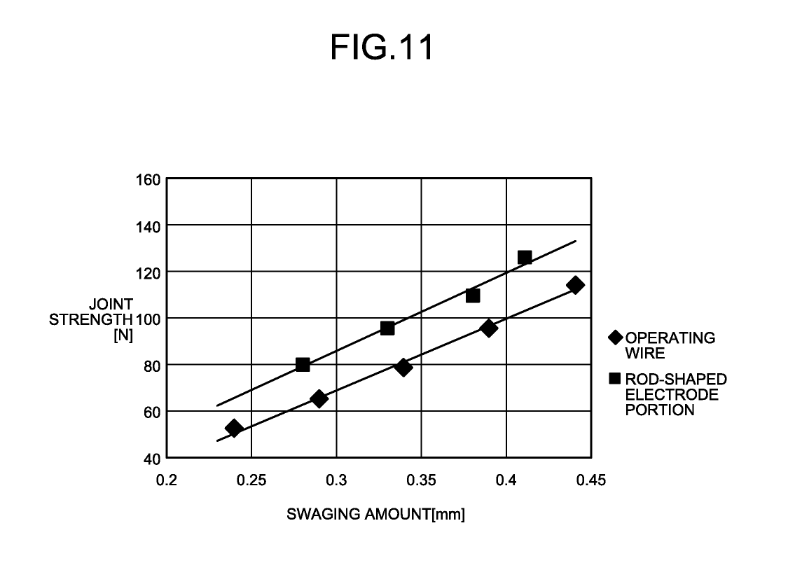

The joint strength between the rod-shaped electrode portion 12a and the coupling member 14 and the joint strength between the operating wire 13 and the coupling member 14 can be adjusted by the swaging amount. FIG. 11 is a graph illustrating a relationship between the swaging amount and the joint strength in the connection structure according to the first embodiment. To obtain this graph, a joint body is produced by joining the rod-shaped electrode portion 12a and the coupling member 14 together and a joint body is produced by joining the operating wire 13 and the coupling member 14 together while changing the swaging amount step-by-step, a tensile test is conducted on the produced joint bodies, and the strength at the time of joint breaking is plotted as the joint strength. As illustrated in FIG. 11, the joint strength at each joint body increases in proportion to the swaging amount.

Here, by obtaining the swaging amount from the joint strength required for each of the joint portion between the rod-shaped electrode portion 12a and the coupling member 14 and the joint portion between the operating wire 13 and the coupling member 14 and by performing each swaging process according to the swaging amount, joining between the rod-shaped electrode portion 12a and the coupling member 14 and between the operating wire 13 and the coupling member 14 can be achieved, respectively, with intended strengths.

Here, in the high frequency knife 1, the joining conditions are set so that each of the joint portion between the rod-shaped electrode portion 12a and the coupling member 14 and the joint portion between the operating wire 13 and the coupling member 14 respectively achieve sufficient joint strengths for a load exerted during a typical endoscopic procedure and that the joint strength between the electrode portion 12 and the coupling member 14 is higher than the joint strength between the operating wire 13 and the coupling member 14. As a matter of course, the strength of each of the electrode portion 12 and the operating wire 13 as a single body is assumed to be higher than the joint strength described above.

Specifically, in a case where a threshold of the load applied to the electrode portion 12 when an excessive operation is performed on the high frequency knife 1 is set to 60 N, from the graph illustrated in FIG. 11, a joint strength of 60 N can be obtained by setting the swaging amount of the operating wire 13 and the coupling member 14 to about 0.27 mm. In a case where the joint strength between the rod-shaped electrode portion 12a and the coupling member 14 is set to 120 N including an additional margin to the strength of the threshold, the swaging amount of the rod-shaped electrode portion 12a and the coupling member 14 may be set to about 0.4 mm.

In practice, it is preferable to determine the swaging amount based on the method of setting the joint strength described above by considering various factors of change in each swaging process when the rod-shaped electrode portion 12a and the coupling member 14 are joined together and the operating wire 13 to the coupling member 14 are joined together, while verifying whether or not the intended required quality can be achieved even in a case where such factors of change occur.

As described above, in the first embodiment of the present invention, when the rod-shaped electrode portion 12a and the operating wire 13 are connected by the coupling member 14, swaging is performed by setting the swaging conditions so that the joint strength between the electrode portion 12 and the coupling member 14 is higher than the joint strength between the operating wire 13 and the coupling member 14. Therefore, even in a case where an excessive operation is performed on the high frequency knife 1 and a load higher than the load applied during the typical endoscopic procedure is applied to the electrode portion 12, the joint portion between the operating wire 13 and the coupling member 14 is broken first, and the electrode portion 12 remains on the insertion portion 10 side with the electrode portion 12 and the coupling member 14 being joined together. Accordingly, the electrode portion 12 can be prevented from coming off.

Modification

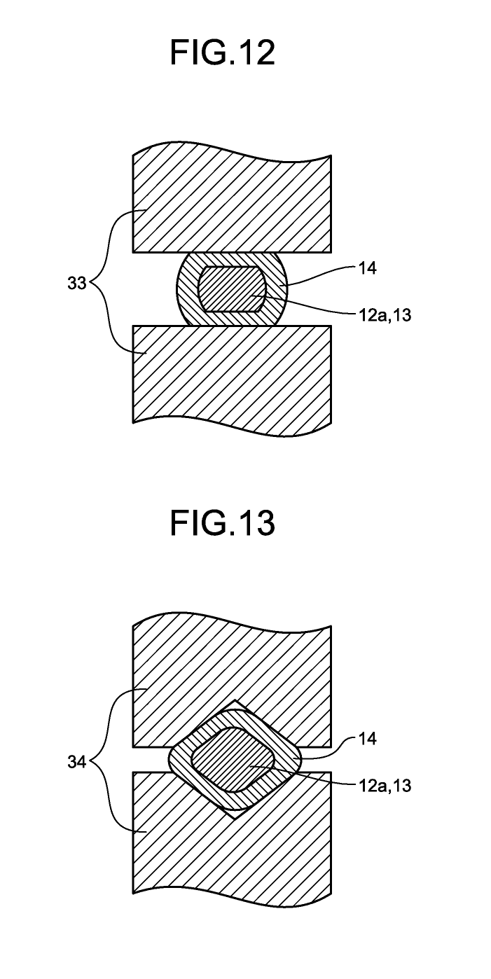

Next, a modification of the first embodiment of the present invention will be described. FIGS. 12 and 13 are sectional views illustrating swaging dies which can be used in first embodiment of the present invention.

In the first embodiment, the exemplary 4-indent swaging tool is used in the swaging process. However, a tool to be used in the swaging process is not limited thereto. For example, the number of swaging dies 30 (see FIGS. 8A and 8B) for pressing the coupling member 14 is not limited to four as long as the number is two or more.

Furthermore, the shape of the swaging die is not limited to a shape that is brought into contact with the outer periphery of the coupling member 14 in a convex shape like the swaging die 30 illustrated in FIGS. 8A and 8B. For example, as illustrated in FIG. 12, joining may be performed by using a pair of swaging dies 33 each having a flat surface to abut on the outer periphery of the coupling member 14, and by causing the coupling member 14, and the rod-shaped electrode portion 12a and the operating wire 13 inserted into the coupling member 14 to be plastically deformed into a flat shape conforming the shape of the swaging dies 33.

Alternatively, as illustrated in FIG. 13, joining may be performed by using a pair of swaging dies 34 each having a bent surface to abut on the outer periphery of the coupling member 14, and by plastically deforming the coupling member 14, and the rod-shaped electrode portion 12a and the operating wire 13 inserted thereinto into a substantially rhomboid shape conforming the shape of the swaging dies 34.

### Second Embodiment

Next, a second embodiment of the present invention will be described. The configuration of an endoscopic treatment tool (high frequency knife) to which a connection structure according to the second embodiment is applied is generally the same as that in first embodiment (see FIG. 1), and the shape of a coupling member that connects an electrode portion 12 to an operating wire 13 is different from that of the first embodiment.

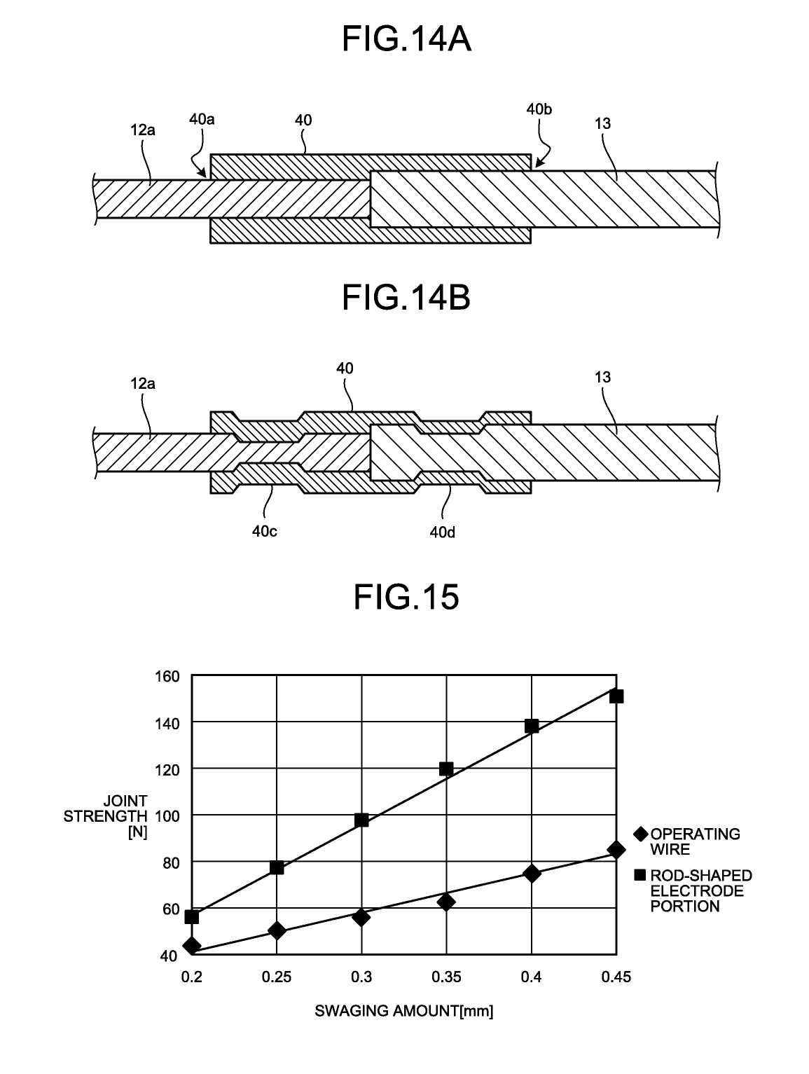

FIGS. 14A and 14B are schematic views for describing the connection structure and a connection method according to the second embodiment. FIG. 14A illustrates a state in which a rod-shaped electrode portion 12a and the operating wire 13 are inserted into a coupling member 40 used in the second embodiment (before connection). FIG. 14B illustrates a state in which the rod-shaped electrode portion 12a, the operating wire 13, and the coupling member 40 are integrated together by swaging the coupling member 40 (after connection).

As illustrated in FIG. 14A, a coupling member 40 is a substantially cylindrical member made of a conductive material such as stainless steel (for example, SUS304). Both ends of the coupling member 40 are respectively provided with an electrode insertion portion 40a into which the rod-shaped electrode portion 12a is configured to be inserted and a wire insertion portion 40b into which the operating wire 13 is configured to be inserted. The electrode insertion portion 40a and the wire insertion portion 40b may or may not communicate with each other inside the coupling member 40. Preferably, the two may communicate with each other, and the end face of the rod-shaped electrode portion 12a and the end face of the operating wire 13 may abut on each other.

The dimensions of the coupling member 40 are appropriately determined according to the dimensions of the rod-shaped electrode portion 12a and the operating wire 13. In the following description, as an example, a case where the rod-shaped electrode portion 12a has an outer diameter of about 0.4 mm and a length of about 10 mm and the operating wire 13 has an outer diameter of about 0.5 mm will be described. In this case, when the coupling member 40 is a rod material or tube material having a length of about 5 mm and a uniform outer diameter of about 0.85 mm, the electrode insertion portion 40a having an inner diameter of about 0.43 mm and a length of 2.5 mm and the wire insertion portion 40b having an inner diameter of about 0.53 mm and a length of about 2.5 mm are formed. In this case, the peripheral wall of the electrode insertion portion 40a and the peripheral wall of the wire insertion portion 40b have different thicknesses, which are about 0.21 mm and about 0.16 mm, respectively.

In the second embodiment, as an example, the outer diameters of the electrode insertion portion 40a and the wire insertion portion 40b of the coupling member 40 are set to the same dimensions; however, the outer diameters thereof may alternatively be set to different dimensions.

In order to connect the electrode portion 12 to the operating wire 13, a process of inserting the rod-shaped electrode portion 12a into the electrode insertion portion 40a of the coupling member 40 and swaging the resultant (hereinafter, referred to as electrode swaging process) and a process of inserting the operating wire 13 into the wire insertion portion 40b and swaging the resultant (hereinafter, referred to as wire swaging process) are sequentially performed. As a result, indents 40c and 40d (see FIG. 14B) are formed on the outer periphery of the coupling member 40. Either the electrode swaging process or the wire swaging process may be performed first. The details of each swaging process are the same as those in the first embodiment (see FIGS. 8A and 8B).

In the second embodiment, unlike the first embodiment, the outer diameters are made uniform without providing a stepped portion (see the tapered portion 14d illustrated in FIGS. 7A and 7B) on the outer periphery of the coupling member 40, while the dimensions of the inner diameters of the electrode insertion portion 40a and the wire insertion portion 40b are set to substantially match the diameters of the rod-shaped electrode portion 12a and the operating wire 13, respectively. Therefore, the peripheral wall of the electrode insertion portion 40a is thicker than the peripheral wall of the wire insertion portion 40b.

Here, as the thickness of the peripheral wall increases, the force to plastically deform the coupling member 40 increases. Therefore, even when the swaging amounts are the same, the joint strength between the electrode portion 12 and the coupling member 40 can be made higher than the joint strength between the operating wire 13 and the coupling member 40. Furthermore, by causing the swaging amounts to be the same, the amount of plastic deformation of the rod-shaped electrode portion 12a and the operating wire 13 can be substantially equal to each other. Therefore, unlike the first embodiment, the load applied to the rod-shaped electrode portion 12a can be reduced. Accordingly, the connection method is effective for products with the rod-shaped electrode portion 12a having low strength.

FIG. 15 is a graph illustrating a relationship between the swaging amount and the joint strength in the connection structure according to the second embodiment. To obtain this graph, a joint body is produced by joining the rod-shaped electrode portion 12a and the coupling member 40 together and a joint body is produced by joining the operating wire 13 and the coupling member 40 together while changing the swaging amount step-by-step, a tensile test is conducted on the produced joint bodies, and the strength at the time of joint breaking is plotted as the joint strength. As illustrated in FIG. 15, the joint strength at each joint body increases in proportion to the swaging amount. In addition, the changes in joint strength between the joint body between the rod-shaped electrode portion 12a and the coupling member 40 and the joint body between the operating wire 13 and the coupling member 40 are compared to each other, the former has a higher joint strength and has a higher rate of change in joint strength with respect to the swaging amount.

As for specific swaging conditions, in a case where 60 N is set as a threshold of the load applied to the electrode portion 12 when an excessive operation is performed on the high frequency knife 1 is set to, it is seen from the graph illustrated in FIG. 15 that a joint strength of about 60 N can be obtained by setting the swaging amount of the operating wire 13 and the coupling member 40 to about 0.35 mm. Furthermore, in a case where the swaging amount of the rod-shaped electrode portion 12a and the coupling member 40 is set to the same value (about 0.35 mm), the joint strength between the rod-shaped electrode portion 12a and the coupling member 40 is about 120 N.

In practice, it is preferable to determine the swaging amount based on the method of setting the joint strength described above by considering various factors of change in each swaging process when the rod-shaped electrode portion 12a, the operating wire 13, and the coupling member 40 are joined together, while verifying whether or not the intended required quality can be achieved even in a case where such factors of change occur.

As described above, according to the second embodiment of the present invention, the joining conditions to cause the joint strength between the electrode portion 12 and the coupling member 40 to be higher than the joint strength between the operating wire 13 and the coupling member 40 can be realized by causing the thickness of the peripheral wall of the electrode insertion portion 40a to be larger than the thickness of the peripheral wall of the wire insertion portion 40b and performing the electrode swaging process and the wire swaging process under the same swaging conditions (swaging amount).

### Third Embodiment

The connection method according to the second embodiment can be performed in combination with the first embodiment. Depending on the specifications of the rod-shaped electrode portion 12a and the operating wire 13, when the connection method according to the first embodiment or the second embodiment is independently performed, an intended joint strength may not be obtained. In such a case, it is possible to achieve the required joint strength by combining the connection methods according to the first embodiment and the second embodiment. Specifically, a method of performing, for example, a swaging process by setting an appropriate swaging amount for each of the electrode insertion portion 40a and the wire insertion portion 40b of the coupling member 40 used in the second embodiment may be employed.

For example, in a case where the difference in outer diameter between the rod-shaped electrode portion 12a and the operating wire 13 is small and the outer diameter of the coupling member 40 is made uniform, a target joint strength may not be set only by the variation in the thickness of the peripheral wall of the coupling member 40. In such a case, as in the first embodiment, the swaging amount of the electrode insertion portion 40a may be set to be larger than the swaging amount of the wire insertion portion 40b. Alternatively, even in a case, for example, where the required strength of the joint portion set in the second embodiment is changed, using the connection method according to the second embodiment in combination with the connection method according to the first embodiment is also effective.

The present invention described above is not limited to the first to third embodiments and the modification, and various inventions can be formed by appropriately combining a plurality of elements disclosed in each embodiment described herein. For example, the other embodiments of the invention may be formed by excluding some elements from all the elements described in each embodiment, or may be formed by appropriately combining the elements described in different embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A connection structure for connecting a treatment component to an operating wire using a coupling member in an endoscopic treatment tool, wherein
the treatment component has a rod-shaped proximal end portion,
the coupling member comprises:
a first hole portion into which the rod-shaped proximal end portion is configured to be inserted on one end side of the coupling member; and
a second hole portion into which the operating wire is configured to be inserted on the other end side of the coupling member,
with the rod-shaped proximal end portion inserted into the first hole portion, the rod-shaped proximal end portion and the coupling member are joined together by swaging and plastically deforming at least a part of a peripheral wall of the first hole portion,
with the operating wire inserted into the second hole portion, the operating wire and the coupling member are joined together by swaging and plastically deforming at least a part of a peripheral wall of the second hole portion, and
a joint strength between the rod-shaped proximal end portion and the coupling member is higher than a joint strength between the operating wire and the coupling member.

2. The connection structure according to claim 1, wherein a swaging amount of the peripheral wall of the first hole portion is larger than a swaging amount of the peripheral wall of the second hole portion.

3. The connection structure according to claim 1, wherein a thickness of the peripheral wall of the first hole portion is larger than a thickness of the peripheral wall of the second hole portion.

4. A method for connecting a treatment component having a rod-shaped proximal end portion to an operating wire in an endoscopic treatment tool using a coupling member, the coupling member having, on one end side thereof, a first hole portion into which the rod-shaped proximal end portion is configured to be inserted and having, on the other end side thereof, a second hole portion into which the operating wire is configured to be inserted, the method comprising:
inserting the rod-shaped proximal end portion into the first hole portion, and swaging and plastically deforming at least a part of a peripheral wall of the first hole portion to join the rod-shaped proximal end portion and the coupling member together; and
inserting the operating wire into the second hole portion, and swaging and plastically deforming at least a part of a peripheral wall of the second hole portion to join the operating wire and the coupling member together in such a way that a joint strength between the operating wire and the coupling member is smaller than a joint strength between the rod-shaped proximal end portion and the coupling member,
wherein joining of the rod-shaped proximal end portion and the coupling member together and joining of the operating wire and the coupling member together are performed in an arbitrary order.

5. The method according to claim 4, wherein a swaging amount in swaging the at least the part of the peripheral wall of the first hole portion is larger than a swaging amount in swaging the at least the part of the peripheral wall of the second hole portion.

6. The method according to claim 4, wherein a thickness of the peripheral wall of the first hole portion before joining the rod-shaped proximal end portion and the coupling member together is larger than a thickness of the peripheral wall of the second hole portion before joining the operating wire and the coupling member together.
